(19) United States
(12) Patent Application Publication
(10) Pub. No.: US 2019/0310253 A1
(43) Pub. Date: Oct. 10, 2019

(54) METHOD FOR DIAGNOSING COMPLICATED PARAPNEUMONIC EFFUSION

(71) Applicant: TAOYUAN ARMED FORCES GENERAL HOSPITAL, Taoyuan City (TW)

(72) Inventors: KUO-AN WU, ZHUBEI CITY (TW); CHIA-YU YANG, TAOYUAN CITY (TW); CHIH-CHING WU, NEW TAIPEI CITY (TW); HSI-HSIEN LIN, TAOYUAN CITY (TW)

(21) Appl. No.: 16/003,313

(22) Filed: Jun. 8, 2018

(30) Foreign Application Priority Data
Apr. 10, 2018 (TW) 107112316

(51) Int. Cl. G01N 33/573; G01N 33/68

(52) U.S. Cl. CPC G01N 33/573; G01N 33/6893; G01N 2800/60; G01N 2333/4743; G01N 2333/904; G01N 2800/12

(57) ABSTRACT

A method for diagnosing complicated parapneumonic effusion is revealed. A sandwich enzyme-linked immunosorbent assay (ELISA) is used to measure a level of a target protein in a pleural effusion. Then the level is compared with a preset baseline corresponding to the target protein so as to determine whether the pleural effusion is a complicated parapneumonic effusion (CPPE). The target protein can be further used in combination with conventional CPPE biomarkers to improve sensitivity and specificity in clinical diagnosis of CPPE.

### BACKGROUND OF THE INVENTION

### Field of the Invention

The present invention relates to a method, especially to a method for diagnosing complicated parapneumonic effusion.

### Description of Related Art

The pleural cavity is a closed fluid-filled space between the visceral pleura and the parietal pleura. Pressure therein is always negative under normal conditions. The space contains 5-20 ml of fluid and the function of the fluid is to facilitate the motion of the covered organs by reducing the friction between the two pulmonary pleura.

The pleural effusion is generated by intercostal arteries of parietal pleura and absorbed by the lymph system. The fluid in the pleural cavity may be increased when the pleura are affected by a certain factor and the dynamic balance of the fluid is changed. A pleural effusion is formed when excess fluid accumulates in the pleural cavity.

The pleural effusion can be categorized into a transudate or an exudate. The transudate is mainly resulted from congestive heart failure, liver cirrhosis, hypoalbuminemia, etc. The exudate is caused by tumors, pneumonia, tuberculosis, pulmonary embolism, etc. which lead to the structure change in/or the increasing permeability of endothelium of lymph vessels/or blood vessels.

Accumulation of the exudate on the same side of the pleural cavity caused by pulmonary infection is called parapneumonic effusions (PPE). From the perspective of pathophysiology, PPE can be divided into three stages: exudative stage, fibrinopurulent stage, and organization stage.

At the early stage, the PPE is a relatively-clear and free-flowing exudate, containing neutrophils. The fluid features on that the bacterial culture result is negative with glucose >60 mg/dL, pH>7.20 and lactate dehydrogenase (LDH) lower than three times of the upper limit of normal (usually <1,000 U/L) and a lower number of leukocytes.

At this stage, PPE is generally classified into a “simple” group, uncomplicated parapneumonic effusion (UPPE). They resolve with appropriate antibiotic treatment and require no chest drainage. Yet without effective treatment, the patients may progress to the next stage within several hours.

At the fibrinopurulent stage, the pleural effusion is typically cloud and containing increased number of neutrophils. The bacteria invasion into the pleural space together with the increasing inflammatory reaction causes increasing amount of lactic acid and carbon dioxide, decreased pH, increased glucose metabolism and an elevated lactic dehydrogenase (LDH) concentration. Thereby complicated parapneumonic effusion (CPPE) occur and drainage/or invasive surgery is required for resolution.

In the fibrinopurulent stage, the glucose level is lower than 60 mg/dL, the pleural effusion pH is lower than 7.20, the LDH level is over three times of the upper limit of normal (usually >1,000 U/L).

Now pus accumulates in the pleural effusion and this is so-called empyema. The patients with the complicated parapneumonic effusion, or empyema, require operative treatment and the mortality rate in the patients is 5-30%.

If antibiotics treatment in combination with chest drainage fails to receive good effects, the patient would progress to the organization stage. Fibrin would be deposited in the pleura and the thickened pleura would encapsulate the lung, preventing the lung from expansion and diminishing the compliance.

In clinical practice, a plurality of indicators including pH value, glucose level and LDH concentration in pleural effusion is used for diagnosis and assessment of severity.

However, the pH value of the pleural effusion can be different owing to different measurements and collection methods. The sampled pleural effusion should be analyzed within 4 hours otherwise the pH value is increased due to the release of carbon dioxide from the pleural effusion to air after long term contact with air.

Although the glucose level can be used for diagnosis of CPPE, the glucose level in patients with malignant pleural effusion (MPE), tuberculous pleural effusion (TPE), blood chest and rheumatoid arthritis (RA) may decrease. As an indicator of CPPE, the sensitivity of the glucose level is decreased.

As to LDH level, it can also be used in CPPE diagnosis. Yet patients with TPE or MPE have higher LDE levels. Thus its sensitivity is reduced.

Thus there is room for improvement and there is a need to find out a novel biomarker for development of a diagnostic test for CPPE in clinical practice.

### SUMMARY OF THE INVENTION

Therefore it is a primary object of the present invention to provide a method for diagnosing complicated parapneumonic effusion in which a level of a target protein in a pleural effusion is measured and then the level measured is compared with a preset baseline for determining whether the pleural effusion is a complicated parapneumonic effusion (CPPE).

It is another object of the present invention to provide a method for diagnosing complicated parapneumonic effusion in which target proteins are used in combination with conventional CPPE biomarkers for improving sensitivity and specificity in clinical diagnosis of CPPE.

In order to achieve the above objects, a method for diagnosing complicated parapneumonic effusion according to the present invention includes the steps of: getting a pleural effusion, measuring a level of a target protein in the pleural effusion and the target protein being selected from the group consisting of bactericidal/permeability-increasing protein (BPI), azurocidin (AZU1), and a combination thereof, comparing the level with a preset baseline to get a comparison result, and determining whether the pleural effusion is a complicated parapneumonic effusion according to the comparison result.

In the step of measuring a level of a target protein in the pleural effusion, the target protein is further selected from the group consisting of neutrophil gelatinase-associated lipocalin (NGAL), calprotectin and a combination thereof.

In the step of getting a pleural effusion, the pleural effusion is drawn from a patient with parapneumonic effusions (PPE).

In the step of measuring a level of a target protein in the pleural effusion, a sandwich enzyme-linked immunosorbent assay (ELISA) is used to measure the level of the target protein.

Moreover, a method for diagnosing complicated parapneumonic effusion according to the present invention includes the steps of: getting a pleural effusion, measuring a bactericidal/permeability-increasing protein (BPI) level in the pleural effusion, comparing the BPI level with a first preset baseline, measuring a lactate dehydrogenase (LDH) level in the pleural effusion when the BPI level is lower than the first preset baseline, comparing the lactate dehydrogenase (LDH) level with a second preset baseline; and determining the pleural effusion to be a complicated parapneumonic effusion when the lactate dehydrogenase (LDH) level is higher than the second preset baseline.

After the step of comparing the bactericidal/permeability-increasing protein (BPI) level with a first preset baseline, the method further includes a step of determining the pleural effusion to be a complicated parapneumonic effusion when the bactericidal/permeability-increasing protein (BPI) level is higher than the first preset baseline.

In the step of comparing the bactericidal/permeability-increasing protein (BPI) level with a first preset baseline, the first preset baseline is 10 ng/ml.

In the step of comparing the lactate dehydrogenase (LDH) level with a second preset baseline, the second preset baseline is 1000 U/L.

In the step of getting a pleural effusion, the pleural effusion is drawn from a patient with parapneumonic effusions (PPE).

In the step of measuring a bactericidal/permeability-increasing protein (BPI) level in the pleural effusion, a sandwich ELISA is used to measure the BPI level.

In the step of measuring a lactate dehydrogenase (LDH) level in the pleural effusion, an enzymatic reaction method is used to measure the lactate dehydrogenase (LDH) level in the pleural effusion.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a flow chart showing steps of an embodiment according to the present invention;

FIG. 2A-2D are schematic drawings showing test results of an embodiment according to the present invention;

FIG. 2E is a chart showing statistical analysis of test results of an embodiment according to the present invention;

FIG. 3 is a flow chart showing steps of another embodiment according to the present invention; and

FIG. 4 is another chart showing steps of another embodiment according to the present invention.

### DETAILED DESCRIPTION OF THE PREFERRED EMBODIMENT

In order to learn features and functions of the present invention, please refer to the following embodiments and related descriptions.

The biomarkers ever been reported now are not better than those conventional biomarkers related to complicated parapneumonic effusion (CPPE) and unable to be used in assistance with the conventional CPPE related biomarkers. Thereby the present invention provides a method for diagnosing complicated parapneumonic effusion and able to solve the problems of the conventional techniques.

The features, related structure and the method of the present invention are described in details in the following embodiments.

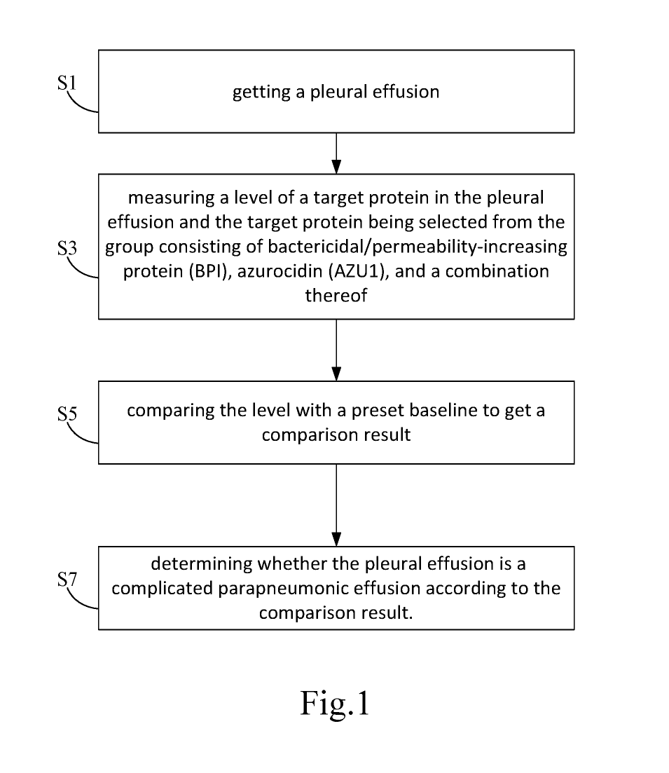

Refer to FIG. 1, a method for diagnosing complicated parapneumonic effusion according to the present invention includes the following steps.

S1: getting a pleural effusion;

S3: measuring a level of a target protein in the pleural effusion and the target protein being selected from the group consisting of bactericidal/permeability-increasing protein (BPI), azurocidin (AZU1), and a combination thereof.

S5: comparing the level with a preset baseline to get a comparison result; and

S7: determining whether the pleural effusion is a complicated parapneumonic effusion according to the comparison result.

As shown in the step S1, getting a pleural effusion. The pleural effusion is sampled from a patient with parapneumonic effusions (PPE). The patient is diagnosed with pneumonia in combination with PPE after chest X-way and ultrasound examinations. The pleural effusion is drawn from the patient with PPE under sonographic guidance.

Then refer to the step S3, measuring a level of a target protein in the pleural effusion. The target protein is selected from the group consisting of bactericidal/permeability-increasing protein (BPI) azurocidin (AUZ1), and a combination thereof. The target protein is further selected from the group consisting of neutrophil gelatinase-associated lipocalin (NGAL), calprotectin and a combination thereof.

Bactericidal/permeability-increasing protein (BPI) is a cationic antimicrobial glycoprotein found in polymorphonuclear neutrophils (PMNs). BPI has potent bactericidal activity against grain-negative bacteria and neutralizes endotoxin activities. It's a product of the antibacterial system of microorganisms.

The azurocidin (AZU1) is a protein found in granules of neutrophils. After further research, it is found that AZU1 is involved in various inflammatory responses.

Neutrophil gelatinase-associated lipocalin (NGAL) is a matrix protein of granules in human neutrophils and strongly associated with inflammation, immune response, cell differentiation, apoptosis, tissue remodeling, and development and progression of a plurality of tumors.

Calprotectin is a soluble protein of the cytosol of a neutrophil. Increasing evidence suggests the implication of calprotectin in inflammatory diseases and cancer.

In the step S3, a sandwich enzyme-linked immunosorbent assay (ELISA) is used to measure the level of the target protein. The sandwich ELISA is often used to detect specific protein and the steps are as follows.

1. A microtiter plate is coated with an antibody with specificity and then the plate is washed to remove unbound antigen. 2. At least one sample is added and antigen contained in the sample is captured by the antibody on the plate specifically. 3. The plate is washed to remove the sample. Then the primary antibody specific to the antigen is added and binds to the antigen. 4. The plate is washed to remove unbound primary antibody. Then the enzyme-linked secondary antibody is added and binds to the primary antibody. 5. The plate is washed to remove unbound secondary antibody and a substrate for the enzyme is added to elicit a color change. Use ELISA reader to read the chromogenic substrate.

The materials for the sandwich ELISA in the present invention include BPI (LSBio, WA, USA), AZU1 (Abnova, CA, USA), NGAL (R &D Systems, MN, USA), and calprotectin (R&D Systems, MN, USA). Follow the assay protocol in the kit manual to perform the sandwich ELISA assay and use ELISA reader to read the optical density (OD) at 450 nm. The OD data is compared with standards containing known concentrations of the analyte so as to calculate the concentration of the biomarker protein in the pleural effusion.

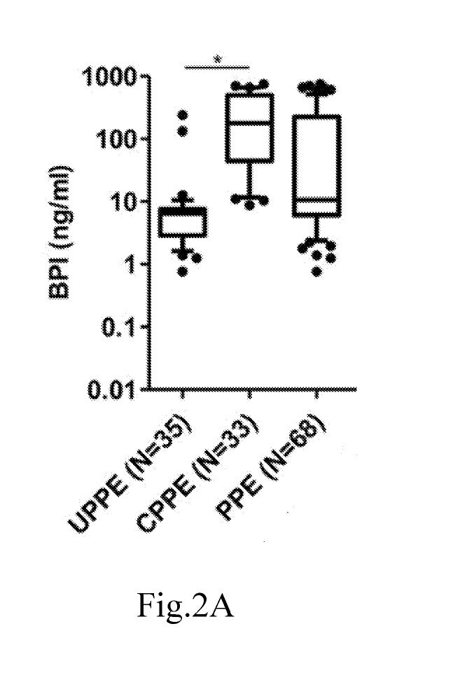

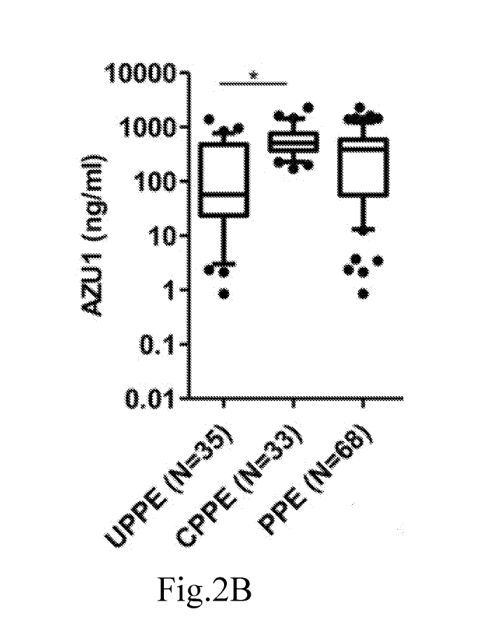

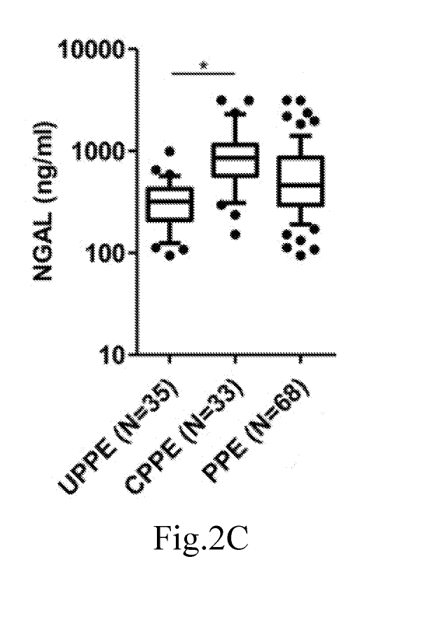

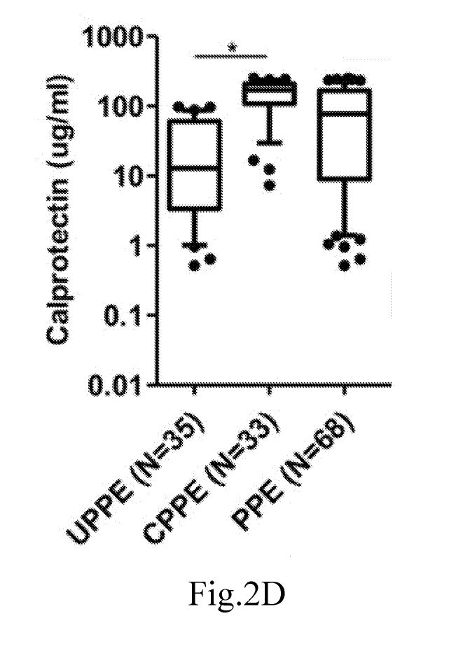

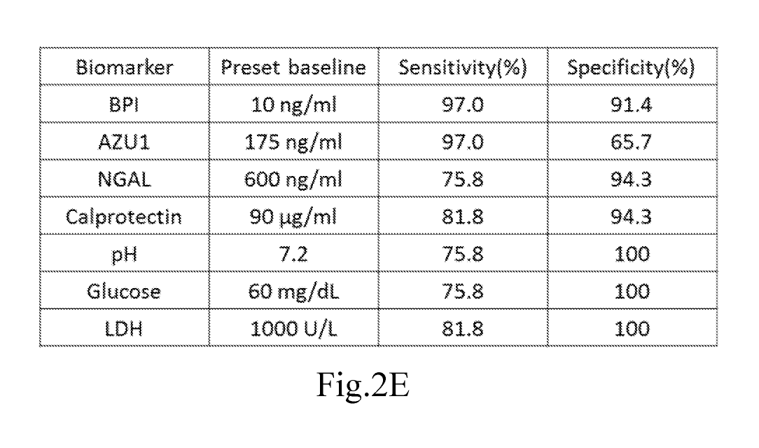

Refer to FIG. 2A-2D, 68 patients with parapneumonic effusions (PPE) are classified into two groups-uncomplicated parapneumonic effusion (UPPE, n=35) and complicated parapneumonic effusion (CPPE, n=33) according to the conventional biomarkers (pH value, glucose level and lactate dehydrogenase (LDH) level). The CPPE is determined once the pH value is smaller than 7.2 (<7.2), the glucose level is lower than 60 mg/dL (<60 mg/dL), and the lactate dehydrogenase (LDH) level is higher than 1000 U/L (>1000 U/L).

The sandwich ELISA is used to measure the level of the target protein of the patients with UPPE and patients with CPPE respectively. Then the preset baseline (basic level) of the respective target protein is set up according to the levels measured and is used for the following tests.

The following test is as shown in the step S5, the level measured is compared with a preset baseline to get a comparison result. The respective target protein has its own preset baseline.

In the step S7, the pleural effusion is determined to be the CPPE according the respective comparison result of different target proteins.

As shown in FIG. 2A, the preset baseline (basic level) is 10 ng/ml when the target protein is the bactericidal/permeability-increasing protein. The pleural effusion is determined to be CPPE when the bactericidal/permeability-increasing protein (BPI) level is higher than the preset baseline (10 ng/ml).

Refer to FIG. 2B, the preset baseline is 175 ng/ml when the target protein is the azurocidin (AZU1). The pleural effusion is determined to be CPPE when the azurocidin (AZU1) level is higher than the preset baseline (175 ng/ml).

Refer to FIG. 2C, the preset baseline is 600 ng/ml when the target protein is the neutrophil gelatinase-associated lipocalin (NGAL). The pleural effusion is determined to be CPPE when the neutrophil gelatinase-associated lipocalin (NGAL) level is higher than the preset baseline (600 ng/ml).

As shown in FIG. 2D, the preset baseline is 90 μg/ml when the target protein is calprotectin. The pleural effusion is determined to be CPPE when the calprotectin level is higher than the preset baseline (90 μg/ml).

Refer to FIG. 2E, both the preset baseline of the target protein and the basic levels of the conventional biomarkers are confirmed. The preset baseline of the bactericidal/permeability-increasing protein (BPI) is 10 ng/ml while the preset baselines of the azurocidin (AZU1), the neutrophil gelatinase-associated lipocalin (NGAL) and calprotectin are 175 ng/ml, 600 ng/ml and 90 μg/ml respectively. The pleural effusion is determined to be CPPE when the level of the target protein is higher than the preset baseline.

The preset basic levels of the conventional biomarkers including pH value, glucose level and lactate dehydrogenase (LDH) level are 7.2, 60 mg/dL and 1000 U/L respectively. The pleural effusion is determined to be CPPE when the pH value is smaller than 7.2 (<7.2), the glucose level is lower than 60 mg/dL (<60 mg/dL), and the lactate dehydrogenase (LDH) level is higher than 1000 U/L (>1000 U/L).

As shown in the FIG. 2E, the sensitivity and specificity of the bactericidal/permeability-increasing protein (BPI) are the highest among the target proteins. The bactericidal/permeability-increasing protein (BPI) has a sensitivity of 97% and specificity of 91.4%. Its sensitivity is much higher than the conventional biomarkers.

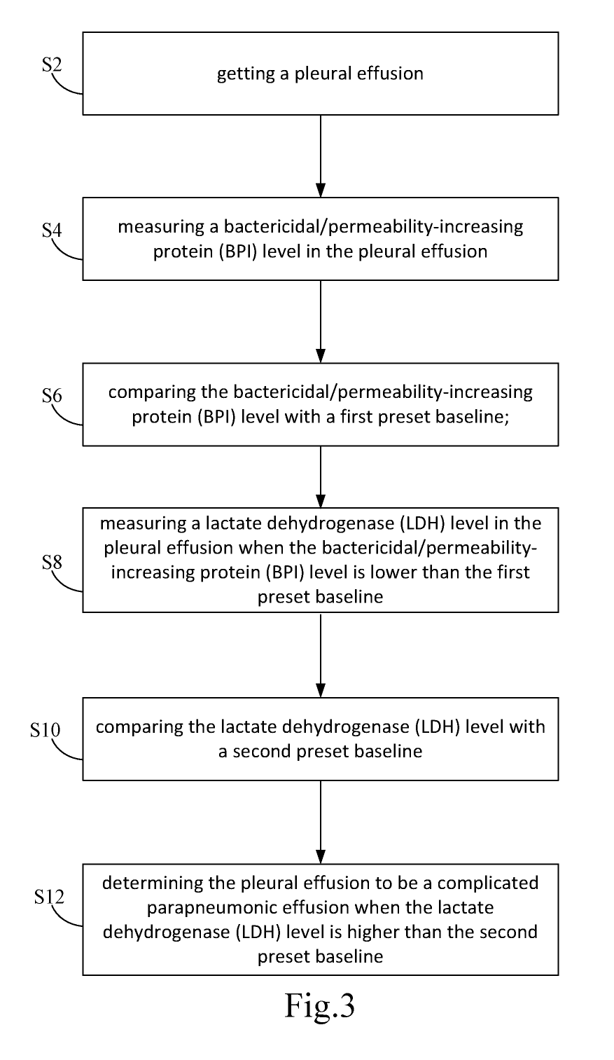

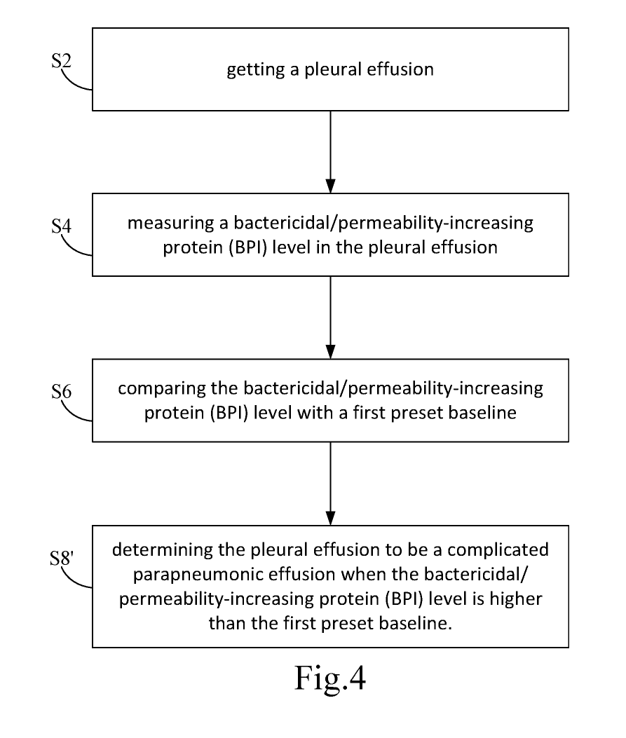

Refer to FIG. 3 and FIG. 4, another embodiment is revealed. In this embodiment, the target protein, the bactericidal/permeability-increasing protein (BPI), is used in combination with the lactate dehydrogenase (LDH) level. As shown in FIG. 3, a method for diagnosing complicated parapneumonic effusion according to the present invention includes the following steps.

S2: getting a pleural effusion;

S4: measuring a bactericidal/permeability-increasing protein (BPI) level in the pleural effusion;

S6: comparing the bactericidal/permeability-increasing protein (BPI) level with a first preset baseline;

S8: measuring a lactate dehydrogenase (LDH) level in the pleural effusion when the bactericidal/permeability-increasing protein (BPI) level is lower than the first preset baseline;

S10: comparing the lactate dehydrogenase (LDH) level with a second preset baseline; and

S12: determining the pleural effusion to be a complicated parapneumonic effusion when the lactate dehydrogenase (LDH) level is higher than the second preset baseline.

As shown in the step S2, getting a pleural effusion. The pleural effusion is obtained from a patient with PPE. The patient is diagnosed with pneumonia in combination with PPE after chest X-way and ultrasound examinations. The pleural effusion is drawn from the patient with PPE under the guidance of ultrasound.

Refer to the step S4, measure a bactericidal/permeability-increasing protein (BPI) level in in the pleural effusion by using a sandwich ELISA.

As shown in the step S6, the bactericidal/permeability-increasing protein (BPI) level measured is compared with a first preset baseline while the first preset baseline is 10 ng/ml.

Refer to the step S8, measure a lactate dehydrogenase (LDH) level in the pleural effusion when the bactericidal/permeability-increasing protein (BPI) level is lower than the first preset basic level. That means an enzymatic reaction method is used to measure the lactate dehydrogenase (LDH) level in the pleural effusion when the bactericidal/permeability-increasing protein (BPI) level is lower than 10 ng/ml.

Refer to FIG. 4, the method further includes a step S8′ after the step S6.

S8′: determining the pleural effusion to be a complicated parapneumonic effusion when the bactericidal/permeability-increasing protein (BPI) level is higher than the first preset baseline.

As shown in the step S8′, the pleural effusion is determined to be a complicated parapneumonic effusion when the bactericidal/permeability-increasing protein (BPI) level is higher than 10 ng/ml.

Next run the step S10, the lactate dehydrogenase (LDH) level measured is compared with a second preset baseline. The second preset baseline is 1000 U/L.

Lastly, as shown in the step S12, the pleural effusion is determined to be a complicated parapneumonic effusion when the lactate dehydrogenase (LDH) level is higher than the second preset basic level. That means the pleural effusion is determined to be a complicated parapneumonic effusion once the lactate dehydrogenase (LDH) level is over 1000 U/L.

The details of the first embodiment of the present invention are described as follows.

A pleural effusion is drawn from a patient with PPE under the guidance of ultrasound and a sandwich ELISA is used to measure a level of a target protein in the pleural effusion. The target protein is selected from the group consisting of BPI, AZU1, NGAL, calprotectin and a combination thereof. Then compare the level of the target protein with a preset baseline to get a comparison result. Different target proteins have different preset baselines respectively. The preset baselines of BPI, AZU1, NGAL, and calprotectin are 10 ng/ml, 175 ng/ml, 600 ng/ml, and 90 μg/ml respectively. The pleural effusion is determined to be CPPE when the comparison result shows that the level of the target protein is higher than the corresponding preset baseline.

The details of another embodiment of the present invention are described as follows.

A pleural effusion is drawn from a patient with PPE under the guidance of ultrasound and a sandwich ELISA is used to measure a BPI level in the pleural effusion. Then compare the BPI level with a first preset baseline to get a comparison result. The first preset baseline is 10 ng/ml. The pleural effusion is determined to be CPPE when the BPI level is higher than 10 ng/ml.

Once the BPI level is lower than 10 ng/ml, an enzymatic reaction method is used to measure a LDH level in the pleural effusion. Next the LDH level is compared with a second preset baseline which is 1000 U/L. The pleural effusion is determined to be CPPE when the LDH level is higher than 1000 U/L.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. A method for diagnosing complicated parapneumonic effusion comprising the steps of:
getting a pleural effusion;
measuring a level of a target protein in the pleural effusion and the target protein being selected from the group consisting of bactericidal/permeability-increasing protein (BPI), azurocidin (AZU1), and a combination thereof;
comparing the level with a preset baseline to get a comparison result; and
determining whether the pleural effusion is a complicated parapneumonic effusion according to the comparison result.

2. The method as claimed in claim 1, wherein the target protein is further selected from the group consisting of neutrophil gelatinase-associated lipocalin (NGAL), calprotectin and a combination thereof in the step of measuring a level of a target protein in the pleural effusion.

3. The method as claimed in claim 1, wherein the pleural effusion is drawn from a patient with parapneumonic effusions (PPE) in the step of getting a pleural effusion.

4. The method as claimed in claim 1, wherein a sandwich enzyme-linked immunosorbent assay (ELISA) is used to measure the level of the target protein in the step of measuring a level of a target protein in the pleural effusion.

5. A method for diagnosing complicated parapneumonic effusion comprising the steps of:
getting a pleural effusion;
measuring a bactericidal/permeability-increasing protein (BPI) level in the pleural effusion;
comparing the bactericidal/permeability-increasing protein (BPI) level with a first preset baseline;
measuring a lactate dehydrogenase (LDH) level in the pleural effusion when the bactericidal/permeability-increasing protein (BPI) level is lower than the first preset baseline;
comparing the lactate dehydrogenase (LDH) level with a second preset baseline; and
determining the pleural effusion to be a complicated parapneumonic effusion when the lactate dehydrogenase (LDH) level is higher than the second preset baseline.

6. The method as claimed in claim 5, wherein the method further includes a step of determining the pleural effusion to be a complicated parapneumonic effusion when the bactericidal/permeability-increasing protein (BPI) level is higher than the first preset baseline after the step of comparing the bactericidal/permeability-increasing protein (BPI) level with a first preset baseline.

7. The method as claimed in claim 5, wherein the first preset baseline is 10 ng/ml.

8. The method as claimed in claim 6, wherein the first preset baseline is 10 ng/ml.

9. The method as claimed in claim 5, wherein the second preset baseline is 1000 U/L in the step of comparing the lactate dehydrogenase (LDH) level with a second preset baseline.

10. The method as claimed in claim 5, wherein the pleural effusion is drawn from a patient with parapneumonic effusion (PPE) in the step of getting a pleural effusion.

11. The method as claimed in claim 5, wherein a sandwich ELISA is used to measure the bactericidal/permeability-increasing protein (BPI) level in the step of measuring a bactericidal/permeability-increasing protein (BPI) level in the pleural effusion.

12. The method as claimed in claim 5, wherein an enzymatic reaction method is used to measure the lactate dehydrogenase (LDH) level in the pleural effusion in the step of measuring a lactate dehydrogenase (LDH) level in the pleural effusion.
